(19) United States
(12) Patent Application Publication
(10) Pub. No.: US 2003/0139470 A1
(43) Pub. Date: Jul. 24, 2003

(54) 3,4-dihydroxybenzyl-substituted carbonic acid derivatives and the use thereof as antioxidants

(76) Inventors: Jakob Peter Ley, Holzminden (DE); William Johncock, H?ouml;xter (DE); Roland Langner, Bevern (DE)

Correspondence Address: BAYER POLYMERS LLC, 100 BAYER ROAD, PITTSBURGH, PA, 15205, US

(21) Appl. No.: 10/312,078

(22) PCT Filed: Jun. 11, 2001
(86) PCT No.: PCT/EP01/06568

(30) Foreign Application Priority Data
Jun. 23, 2000 (DE) 100 30 891.0

(51) Int. Cl. A61K 31/325; C07C 271/06

(52) U.S. Cl. 514/487; 560/29

(57) ABSTRACT

The invention relates to 3,4-dihydroxybenzyl-substituted carbonic acid derivatives, to their production, and to their use as antioxidants or free-radical scavengers, especially in cosmetic and pharmaceutical preparations and in foodstuffs and stimulants, to protect cells and tissues from the harmful effects of radicals and reactive oxygen compounds that accelerate aging. The invention further relates to cosmetic and pharmaceutical preparations and to foodstuffs and stimulants that comprises the inventive 3,4-dihydroxybenzyl-substituted carbonic acid derivatives.

The invention relates to 3,4-dihydroxybenzyl-substituted carbonic acid derivatives, to the preparation thereof and to the use thereof as antioxidants and/or free-radical scavengers, in particular in cosmetic and/or pharmaceutical preparations for protecting cells and tissue of humans and animals against the harmful effects of free radicals and reactive oxygen compounds which accelerate aging. The invention further relates to cosmetic and/or pharmaceutical preparations, and to foods and/or luxury products comprising these 3,4-dihydroxybenzyl-substituted carbonic acid derivatives.

Ultraviolet light, in particular ultraviolet light in the range from 290 to 400 nm, triggers photooxidative processes on and in the skin, in particular of humans and animals, various reactive oxygen compounds or free radicals being formed starting from oxygen. These may, for example, harm or destroy intracellular molecules and thus weaken the cell in its vitality or even cause it to die. In addition, the reactive oxygen compounds or free radicals can also damage the intracellular molecules or structures. In the case of skin, the lipid layer, which serves as a barrier against the environment, and also the sebum may be destroyed by oxidative processes. A major constituent of the vital sebum is considerably unsaturated squalene.

Substances and preparations of these substances which, in physiological systems, support the natural defence mechanisms against free radicals and reactive oxygen compounds and/or, as antioxidants or free radical scavengers, protect the lipid layer of the skin against oxidative processes, safeguard the skin from destruction and aging. Such substances also protect oxygen-sensitive materials, such as oxidation-sensitive constituents of cosmetics, pharmaceuticals or foods and/or luxury products.

Antioxidants (oxidation inhibitors) are usually organic compounds which inhibit or prevent the undesired changes in the substances to be protected caused by oxygen effects and also oxidative processes (Römpp Lexikon Chemie 10th edition, 229 (1996)). Many antioxidants also function as free-radical scavengers and/or as complexing agents for heavy metal ions.

The object of the present invention is to make available antioxidants with a strong specific free-radical scavenging and/or antioxidative action for use in cosmetic and pharmaceutical preparations, foods and luxury products, and for protecting cells and tissue of humans and animals.

We have now found novel 3,4-dihydroxybenzyl-substituted carbonic acid derivatives of the general formula I,

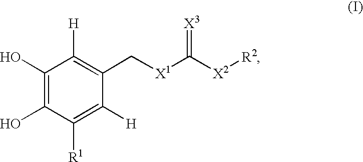

where

X¹, X² and X³, independently of one another, are oxygen atoms, sulfur atoms or groups —N—R³,

and

R¹ is a hydrogen atom, a lower alkyl, lower alkenyl, 1-oxo-lower alkyl or 1-oxo-lower alkenyl group having 1 to 5 carbon atoms or a group —O—R⁴, in which R⁴ is a hydrogen atom, a lower alkyl, lower alkenyl, 1-oxo-lower alkyl or 1-oxo-lower alkenyl group having 1 to 5 carbon atoms,

and

R² is a hydrogen atom, a branched or unbranched, cyclic or stretched alkyl or alkenyl group having 1 to 22 carbon atoms, where one or more of the carbon atoms may be replaced by oxygen atoms, or a ring-substituted arylalkyl group having 7 to 15 carbon atoms, with the proviso that the ring-substituted arylalkyl group does not contain halogen atoms,

and

R³ is a hydrogen atom, a branched or unbranched, cyclic or stretched alkyl or alkenyl group having 1 to 22 carbon atoms, where one or more of the carbon atoms may be replaced by oxygen atoms.

EP A 0,068,592 and U.S. Pat. No. 4,443,473 describe aralkyl, alkyl or alkenyl carbamates and thiocarbamates of hydroxy-substituted benzylamines and corresponding pharmaceutical preparations, where the alkyl radicals can contain 3 to 12 carbon atoms. However, these specifications do not list any 3,4-dihydroxybenzyl-substituted carbonic acid derivatives. The synthesis of N-(2-(4-chlorophenyl)ethyl)-N′-(3,4-dihydroxybenzyl)thiourea has been described in J. Med. Chem., 1994, 37, pages 1942 to 1954. Further 3,4-dihydroxybenzyl-substituted carbonic acid derivatives are not known. Just as little with regard to the antioxidative or free-radical scavenging property of the compounds emerges from said specifications.

The novel 3,4-dihydroxybenzyl-substituted carbonic acid derivatives have, in comparison to known antioxidants, an excellent protective action on the skin of humans and animals against destruction and aging.

The invention preferably relates to novel 3,4-dihydroxybenzyl-substituted carbonic acid derivatives of the general formula (Ia),

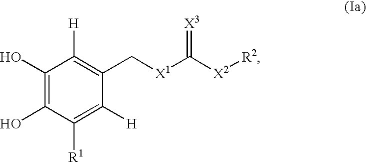

where

R¹ is a hydrogen atom, a lower alkyl, lower alkenyl, 1-oxo-lower alkyl or 1-oxo-lower alkenyl group having 1 to 5 carbon atoms or a group —O—R⁴, in which R⁴ is a hydrogen atom, a lower alkyl, lower alkenyl, 1-oxo-lower-alkyl or 1-oxo-lower-alkenyl group having 1 to 5 carbon atoms,

and

X¹ is —NH, and

X² and X³ are oxygen, and

R² is a hydrogen atom, a methyl, an ethyl or an ethenyl group.

The invention further preferably relates to novel 3,4-dihydroxybenzyl-substituted carbonic acid derivatives of the general formula (Ib),

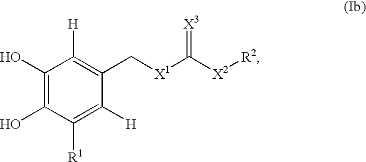

where

R¹ is a hydrogen atom, a lower alkyl, lower alkenyl, 1-oxo-lower alkyl or 1-oxo-lower alkenyl group having 1 to 5 carbon atoms or a group —O—R⁴, in which R⁴ is a hydrogen atom, a lower alkyl, lower alkenyl, 1-oxo-lower alkyl or 1-oxo-lower alkenyl group having 1 to 5 carbon atoms,

and

X¹ is —NH, and

X² and X³ are oxygen, and

R² is a branched or unbranched, cyclic or stretched alkyl or alkenyl group having 3 to 22 carbon atoms, where one or more of the carbon atoms may be replaced by oxygen atoms, or a ring-substituted arylalkyl group having 7 to 15 carbon atoms, with the proviso that the ring-substituted arylalkyl group does not contain halogen atoms.

The invention further preferably relates to novel 3,4-dihydroxybenzyl-substituted carbonic acid derivatives of the general formula (Ic),

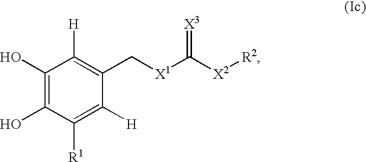

where

R¹ is a hydrogen atom, a lower alkyl, lower alkenyl, 1-oxo-lower alkyl or 1-oxo-lower alkenyl group having 1 to 5 carbon atoms or a group —O—R⁴, in which R⁴ is a hydrogen atom, a lower alkyl, lower alkenyl, 1-oxo-lower alkyl or 1-oxo-lower alkenyl group having 1 to 5 carbon atoms,

X¹, X² and X³, independently of one another, are oxygen atoms, sulfur atoms or groups —NH, with the proviso that X¹ is not —NH at the same time as X² and X³ are oxygen,

and

R² is a branched or unbranched, cyclic or stretched alkyl or alkenyl group having 1 to 22 carbon atoms, where one or more of the carbon atoms may be replaced by oxygen atoms, or a ring-substituted arylalkyl group having 7 to 15 carbon atoms, with the proviso that the ring-substituted arylalkyl group does not contain halogen atoms.

A lower alkyl group consists of 1 to 5 carbon atoms and may, for example, be: methyl, ethyl, 1-propyl, 2-propyl-, 1-butyl, 2-butyl, tert.-butyl, 2-methylprop-1-yl, cyclopropyl, cyclopropylmethyl, 2,2-dimethylcyclopropyl, cyclobutyl, 1-, 2- or 3-pentyl-, 2-methylbut-1-yl, 2-methylbut-2-yl, 3-methylbut-1-yl, 3-methylbut-2-yl or cyclopentyl.

A lower alkenyl group consists of 2 to 5 carbon atoms and may, for example, be: ethenyl, prop-2-en-1-yl, prop-1-en-1-yl, prop-1-en-2-yl, 1- or 2-cyclopropenyl-, but-1-en-1-yl, but-1-en-2-yl, but-1-en-3-yl, but-2-en-1-yl, but-3-en-1-yl, but-2-en-2-yl, 2-methylprop-1-en-1-yl, 2-methylprop-2-en-1-yl, 1,3-butadien-1-yl, 1,3-butadien-2-yl, pent-1-en-1-yl, pent-1-en-2-yl, pent-1-en-3-yl, pent-1-en-4-yl, pent-2-en-1-yl, pent-2-en-2-yl, pent-2-en-3-yl, pent-2-en-4-yl, pent-3-en-1-yl, pent-4-en-1-yl, 1,3-pentadien-1-yl, 1,3-pentadien-2-yl, 1,3-pentadien-3-yl, 2,4-pentadien-2-yl, 2,4-pentadien-1-yl, 1,4-pentadien-1-yl, 1,4-pentadien-2-yl, 1,4-pentadien-3-yl, 1-,2- or 3-cyclopentenyl, 1-,2- or 3-cyclopentadienyl, 3-methylbut-1-en-1-yl, 3-methylbut-1-en-2-yl, 3-methylbut-1-en-3-yl, 3-methylbut-1-en-4-yl, 3-methylbut-2-en-1-yl, 3-methylbut-2-en-2-yl, 3-methylbut-2-en-4-yl, 2-methylbut-1-en-1-yl, 2-methylbut-1-en-3-yl, 2-methylbut-1-en-4-yl, 2-methylidenebut-1-yl, 2-methyl-1,3-butadien-1-yl, 2-methyl-1,3-butadien-3-yl, 2-methyl-1,3-butadien-4-yl, 2-methylidenebut-3-en-1-yl, and the Z and E isomers of the abovementioned radicals which may be possible in each case.

A lower alkyl-1-oxo group consists of 1 to 5 carbon atoms and can, for example, be: formyl, acetyl, propanoyl, butanoyl, 2-methylpropanoyl, pentanoyl, 2- or 3-methylbutanoyl, 2,2-dimethylpropanoyl, cyclopropylcarboxyl.

A 1-oxoalkenyl group can contain 3 to 5 carbon atoms and may, for example, be: prop-2-enoyl, 2-methylprop-2-enoyl, 2-ethylprop-2-enoyl, E- or Z-2-butenoyl, 3-butenoyl, E- or Z-2-methylbut-2-enoyl, E- or Z-3-methylbut-2-enoyl, Z- or E-2-pentenoyl, Z- or E-3-pentenoyl.

An unbranched, branched or cyclic alkyl group can contain 1 to 22, preferably 1 to 20, especially preferably 1 to 18, carbon atoms. Examples which may be mentioned are: methyl, ethyl, 1-propyl, 2-propyl-, 1-butyl, 2-butyl, tert.-butyl, 2-methyl, 2-methylprop-1-yl, cyclopropyl, cyclopropylmethyl, 2,2-dimethylcyclopropyl, cyclobutyl, cyclopentyl, cyclohexyl and various positional isomers of methylpentyl, menthyl, 1-pentyl, 1-hexyl, 1-heptyl-, 1-octyl, 2-ethylhexyl, 1-nonyl, 1-decyl, 1-undecyl, 1-dodecyl, 1-tridecyl, 1-tetradecyl, 1-pentadecyl, 1-hexadecyl, 1-heptadecyl and 1-octadecyl.

An unbranched, branched or cyclic alkenyl group can contain 2 to 22, preferably 2 to 20, especially preferably 2 to 18 carbon atoms. Examples which may be mentioned are: ethenyl, prop-2-en-1-yl, prop-1-en-1-yl, prop-1-en-2-yl, 1- or 2-cyclopropenyl-, but-1-en-1-yl, but-1-en-2-yl, but-1-en-3-yl, but-2-en-1-yl, but-3-en-1-yl, but-2-en-2-yl, 2-methylprop-1-en-1-yl, 2-methylprop-2-en-1-yl, 1,3-butadien-1-yl, 1,3-butadien-2-yl, pent-1-en-1-yl, pent-1-en-2-yl, pent-1-en-3-yl, pent-1-en-4-yl, pent-2-en-1-yl, pent-2-en-2-yl, pent-2-en-3-yl, pent-2-en-4-yl, pent-3-en-1-yl, pent-4-en-1-yl, 1,3-pentadien-1-yl, 1,3-pentadien-2-yl, 1,3-pentadien-3-yl, 2,4-pentadien-2-yl, 2,4-pentadien-1-yl, 1,4-pentadien-1-yl, 1,4-pentadien-2-yl, 1,4-pentadien-3-yl, 1-,2- or 3-cyclopentenyl, 1-,2- or 3-cyclopentadienyl, 3-methylbut-1-en-1-yl, 3-methylbut-1-en-2-yl, 3-methylbut-1-en-3-yl, 3-methylbut-1-en-4-yl, 3-methylbut-2-en-1-yl, 3-methylbut-2-en-2-yl, 3-methylbut-2-en-4-yl, 2-methylbut-1-en-1-yl, 2-methylbut-1-en-3-yl, 2-methylbut-1-en-4-yl, 2-methylidenebut-1-yl, 2-methyl-1,3-butadien-1-yl, 2-methyl-1,3-butadien-3-yl, 2-methyl-1,3-butadien-4-yl, 2-methylidenebut-3-en-1-yl, the various positional and double-bond isomers of the heptenyl, the octenyl, the nonenyl, the decenyl, the dodecenyl, the tetradecenyl, the hexadecenyl, the octadecenyl radical, Z-hexadeca-9-en-1-yl, Z-octadeca-9-en-1-yl, Z,Z-octadeca-9,12-dien-1-yl, Z,Z,Z-octadeca-9,12,15-trien-1-yl, E-octadeca-9-en-1-yl.

A ring-substituted arylalkyl group can consist of 7 to 15 carbon atoms, preferably of 7 to 8 carbon atoms, with the proviso that at least one further substituent on the aromatic moiety is not hydrogen. Particular preference is given to a ring-substituted benzyl, a 2- or 1-phenylethyl group.

Substituents of the ring-substituted arylalkyl group may, for example, be: hydrogen atoms, lower alkyl, hydroxyl, lower alkyloxy, thio, lower alkylthio, amino, lower alkylamino, di-lower alkylamino, phosphate, lower alkylphosphate, di-lower alkylphosphate, sulfonic acid, lower alkylsulfonate, sulfonamide, di-lower alkylsulfonamide or lower alkylsulfonamide radicals. Particular preference is given to hydrogen atoms, lower alkyl, hydroxyl and lower alkyloxy radicals, with the proviso that the substituents are not halogen.

Particular preference is given to 3,4-dihydroxybenzyl-substituted carbonic acid derivatives of the general formula (Ia),

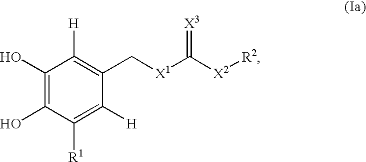

where

R¹ is a hydrogen atom, a lower alkyl group having 1 to 5 carbon atoms or a group —O—R⁴, in which R⁴ is a hydrogen atom or a lower alkyl group having 1 to 5 carbon atoms,

and

X¹ is —NH, and

X² and X³ are oxygen, and

R² is a hydrogen atom, a methyl, an ethyl or an ethenyl group.

Particular preference is given to 3,4-dihydroxybenzyl-substituted carbonic acid derivatives of the general formula (Ib),

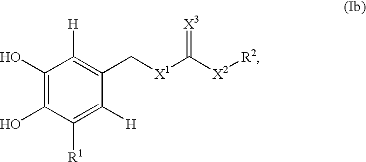

where

R¹ is a hydrogen atom, a lower alkyl group having 1 to 5 carbon atoms or a group —O—R⁴, in which R⁴ is a hydrogen atom or a lower alkyl group having 1 to 5 carbon atoms,

and

X¹ is —NH, and

X² and X³ are oxygen, and

R² is a branched or unbranched, cyclic or stretched alkyl or alkenyl group having 3 to 20 carbon atoms, where one or more of the carbon atoms may be replaced by oxygen atoms, or a ring-substituted arylalkyl group having 7 to 8 carbon atoms, with the proviso that the ring-substituted arylalkyl group does not contain halogen atoms.

Particular preference is given to 3,4-dihydroxybenzyl-substituted carbonic acid derivatives of the general formula (Ic),

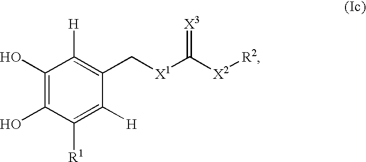

where

X¹ and X², independently of one another, are oxygen atoms, sulfur atoms or —N—H,

and

X³ is an oxygen atom or a sulfur atom, with the proviso that X¹ is not —NH at the same time as X² and X³ are oxygen

and

R¹ is a hydrogen atom, a lower alkyl group having 1 to 5 carbon atoms or a group —O—R⁴, in which R⁴ is a hydrogen atom or a lower alkyl group having 1 to 5 carbon atoms,

and

R² is a branched or unbranched, cyclic or stretched alkyl or alkenyl group having 1 to 20 carbon atoms, where one or more of the carbon atoms may be replaced by oxygen atoms, or a ring-substituted arylalkyl group having 7 to 8 carbon atoms, with the proviso that the ring-substituted arylalkyl group does not contain halogen atoms.

Particular preference is given to 3,4-dihydroxybenzyl-substituted carbonic acid derivatives of the general formula (Ia),

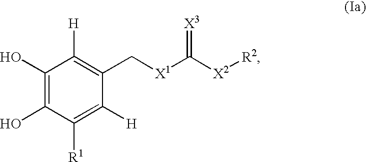

where

R¹ is a hydrogen atom, a methoxy or a hydroxyl group,

and

X¹ is —NH, and

X² and X³ are oxygen, and

R² is a methyl, an ethyl or an ethenyl group.

Particular preference is given to 3,4-dihydroxybenzyl-substituted carbonic acid derivatives of the general formula (Ib),

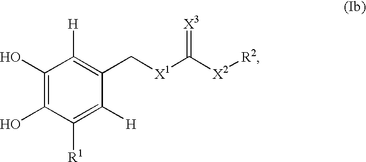

where

R¹ is a hydrogen atom, a methoxy or a hydroxyl group, and

X¹ is —NH, and

X² and X³ are oxygen, and

R² is a branched or unbranched, cyclic or stretched alkyl group having 3 to 18 carbon atoms, a branched or unbranched, cyclic or stretched alkenyl group having 3 to 18 carbon atoms or a ring-substituted benzyl, ring-substituted 2-phenylethyl or ring-substituted 1-phenylethyl radical, with the proviso that the ring-substituted arylalkyl group does not contain halogen atoms.

Particular preference is given to 3,4-dihydroxybenzyl-substituted carbonic acid derivatives of the general formula (Ic),

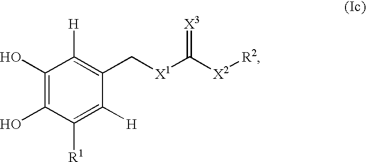

where

R¹ is a hydrogen atom, a methoxy or a hydroxyl group,

and

X¹ is a group —N—H, and

X² is an oxygen atom, sulfur atom or —N—H, and

X³ is an oxygen atom or a sulfur atom, with the proviso that X² and X³ are not oxygen at the same time, and

R² is a branched or unbranched, cyclic or stretched alkyl group having 1 to 18 carbon atoms, a branched or unbranched, cyclic or stretched alkenyl group having 2 to 18 carbon atoms or a ring-substituted benzyl, ring-substituted 2-phenylethyl or ring-substituted 1-phenylethyl radical, with the proviso that the ring-substituted arylalkyl group does not contain halogen atoms.

Individual compounds according to the invention which may be mentioned are, for example,

N-(3,4-dihydroxybenzyl)-O-methylurethane

N-(3,4-dihydroxybenzyl)-O-[(1R,3R,4S)-menthyl]urethane

O-methyl-N-(3,4,5-trihydroxybenzyl)urethane

N-(3,4-dihydroxybenzyl)-O-hexylurethane

N-(3,4-dihydroxybenzyl)-O-(2-ethylhexyl)urethane

N-(3,4-dihydroxybenzyl)-N′-hexylthiourea

N,N′-bis(3,4-dihydroxybenzyl)urea.

If, in the 3,4-dihydroxybenzyl-substituted carbonic acid derivatives according to the invention, at least one of the radicals X¹, X² and X³ is —N—H, the compounds according to the invention may also be present in the form of their tautomers.

Surprisingly, it has now been found that the 3,4-dihydroxybenzyl-substituted carbonic acid derivatives according to the invention are very good free-radical scavengers and particularly strong antioxidants. They are preferably suitable as antioxidants for lipids. In particular the 3,4-dihydroxybenzyl-substituted carbonic acid derivatives according to the invention are able to suppress the harmful effects of free radicals and/or oxidative processes which are induced by UV light on and/or in human skin, and to support the natural antioxidative processes.

The 3,4-dihydroxybenzyl-substituted carbonic acid derivatives according to the invention support, in physiological systems of the skin, the hair or the nails, the natural defence mechanisms against free radicals and reactive oxygen compounds and protect, in cosmetics, pharmaceuticals or foods, oxidation-sensitive constituents thereof against autoxidation or photooxidation.

The 3,4-dihydroxybenzyl-substituted carbonic acid derivatives according to the invention can therefore be used in cosmetic or pharmaceutical, in particular cosmetic or dermatological, preparations for protecting cells and tissues of mammals, in particular of humans, against the harmful effect of free radicals and reactive oxygen species. The 3,4-dihydroxybenzyl-substituted carbonic acid derivatives according to the invention can of course also be used in other preparations, for example pharmaceutical preparations, preparations for protecting or influencing plants, in foods or luxury products or preparations for food supplements, or in other products, for example oxidation-sensitive natural or synthetic polymers (e.g. rubber, polyolefins), as antioxidants or free-radical scavengers.

The amount of the 3,4-dihydroxybenzyl-substituted carbonic acid derivatives according to the invention in a cosmetic or pharmaceutical preparation is 0.001% by weight to 10% by weight, preferably 0.001 to 5% by weight, particularly preferably 0.001% by weight to 1% by weight, based on the total weight of the preparation.

The 3,4-dihydroxybenzyl-substituted carbonic acid derivatives according to the invention can be synthesized using methods known per se, by reacting a 3,4-dihydroxybenzyl derivative of the general formula (II)

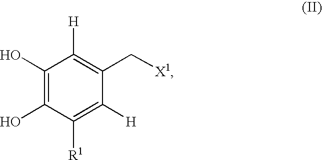

where

X¹ is a group —O—H, —S—H, —NH₂ or —(NH₃)⁺, and

R¹ has the meaning given above

either with an activated carbonic acid derivative of the general formula (III)

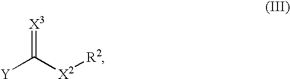

where

X², X³ and R² have the meaning given above and

Y is a halogen atom, a group —N₃, —O—N═C(C₆H₅)CN, —O—R⁵ or —S—R⁵, and

R⁵ may be a lower alkyl, 1-oxo-lower alkyl, lower alkenyl, 1-oxo-lower alkenyl, aryl, arylalkyl, aryl-1-oxoalkyl or lower alkyloxycarbonyl group,

or

with a heterocumulene of the general formula (IV)

X⁴═C═N—R² (IV),

where

X⁴ is an oxygen atom or a sulfur atom and

R² has the meaning given above, or

with phosgene or triphosgene with or without solvent and optionally with the admixing of an auxiliary base and either directly after purification with a compound of the general formula (V)

Z-R² (V),

where

Z is a group —O—H, —S—H, —NH₂ or —(NH₃)⁺ and

R² has the meaning given above

with or without solvent and optionally with the admixing of an auxiliary base.

The particularly preferred 3,4-dihydroxybenzyl-substituted carbonic acid derivatives where X¹ is —N—H, X² and X³ are in each case an oxygen atom and R¹ and R² have the meaning given above can be obtained, for example, by reacting a 3,4-dihydroxybenzylamine of the general formula (II), where X¹ is a group —NH₂ or —(NH₃)⁺ and R¹ has the meaning given above, with a chloroformic ester of the general formula (III) where X² and X³ are oxygen atoms, Y is a chlorine atom and R² has the meaning given above, in a solvent or solvent mixture, preferably chosen from the group consisting of water, acetone, 1,4-dioxane, tetrahydrofuran, various aliphatic esters of aliphatic alcohols (such as, for example, ethyl acetate), chlorine-containing solvents (e.g. chloroform) or aromatic solvents (e.g. benzene, toluene), and advantageously one or more auxiliary bases, preferably chosen from the group of alkali metal hydroxides (e.g. NaOH), alkali metal carbonates (e.g. Na₂CO₃ or NaHCO₃), alkaline earth metal hydroxyides (e.g. Mg(OH)₂), alkaline earth metal oxides (e.g. CaO) or alkaline earth metal carbonates (e.g. CaCO₃), ammonia, aliphatic amines (e.g. triethylamine or diisopropylamine), heterocyclic amines (e.g. pyridine or 4-(N,N-dimethylamino)pyridine) or basic inorganic or organic ion exchangers.

The particularly preferred 3,4-dihydroxybenzyl-substituted carbonic acid derivatives of the general formula (Ic) where X¹ and X² are —N—H and X³ is an oxygen or sulfur atom and R¹ and R² have the meaning given above can be obtained, for example, by reacting a 3,4-dihydroxybenzylamine of the general formula (II), where X¹ is a group —NH₂ or —(NH₃)⁺ and R¹ has the meaning given above, with a heterocumulene of the general formula (IV), where X⁴ and R² have the meanings given above, in a solvent or solvent mixture, preferably chosen from the group consisting of water, acetone, 1,4-dioxane, tetrahydrofuran, various aliphatic esters of aliphatic alcohols (such as, for example, ethyl acetate), chlorine-containing solvents (e.g. chloroform) or aromatic solvents (e.g. benzene), and advantageously one or more auxiliary bases, preferably chosen from the group of alkali metal hydroxides (e.g. NaOH), alkali metal carbonates (e.g. Na₂CO₃ or NaHCO₃), alkaline earth metal hydroxides (e.g. Mg(OH)₂), alkaline earth metal oxides (e.g. CaO) or alkaline earth metal carbonates (e.g. CaCO₃), ammonia, aliphatic amines (e.g. triethylamine or diisopropylamine) or heterocyclic amines (e.g. pyridine or 4-(N,N-dimethylamino)pyridine) or basic inorganic or organic ion exchangers.

Since derivatives substituted on the phenolic hydroxyl groups can also be formed in the processes according to the invention, a saponification step is advantageously connected downstream, in particular a basic saponification with one of the abovementioned bases in the abovementioned solvents or solvent mixtures, preferably water or water-containing solvent mixtures, at temperatures of from 30 to 130° C., preferably 50 to 100° C., in which the phenyl esters are selectively cleaved and the 3,4-dihydroxybenzyl-substituted carbonic acid derivatives of the general formula (Ia) to (Ic) according to the invention are obtained in free form following adjustment of the pH to values of <7, preferably with mineral acids (e.g. hydrochloric acid).

The 3,4-dihydroxybenzyl-substituted carbonic acid derivatives according to the invention can be further purified using methods known to the person skilled in the art, preferably crystallization, chromatographic methods or distillation.

The 3,4-dihydroxybenzyl derivatives of the general formula (II) used are, in particular, 3,4-dihydroxybenzylamine or ammonium salts thereof, 3,4-dihydroxy-5-methylbenzylamine or ammonium salts thereof, 3,4-dihydroxy-5-methoxybenzylamine or ammonium salts thereof, 3,4,5-trihydroxybenzylamine or ammonium salts thereof, 3,4-dihydroxybenzyl alcohol or 3,4,5-trihydroxybenzyl alcohol.

The activated carbonic acid derivatives of the general formula (III) used are preferably methyl chloroformate, ethyl chloroformate, propyl chloroformate, isopropyl chloroformate, n-butyl chloroformate, chloroformic acid, 2-(tert-butoxycarbonyloxyimino)-2-phenylacetonitrile, di-tert-butyl pyrocarbonate, (−)-menthyl chloroformate, n-hexyl chloroformate, 2-ethylhexyl chloroformate or other chloroformic esters of branched or unbranched, stretched or cyclic alkanols or alkenols.

The heterocumulenes of the general formula (IV) are preferably chosen from the group consisting of methyl, ethyl, propyl, isopropyl, n-butyl, isobutyl, sec-butyl, tert-butyl, the various stretched or cyclic isomers of pentyl, hexyl, heptyl and octyl isothiocyanates or the corresponding isocyanates.

The compounds of the general formula (V) are preferably chosen from the group consisting of methyl-, ethyl-, propyl-, isopropyl-, n-butyl-, isobutyl-, sec-butyl-, tert-butyl-, the various stretched or cyclic isomers of pentyl-, hexyl-, heptyl-, ring-substituted benzyl-, 1-phenylethyl- and 2-phenylethylamines, alcohols or thiols.

The present invention also further relates to cosmetic and pharmaceutical preparations, in particular cosmetic and dermatological preparations which comprise the 3,4-dihydroxybenzyl-substituted carbonic acid derivatives according to the invention in an effective amount, alongside other, otherwise customary composition constituents. They comprise 0.001% by weight to 10% by weight, preferably 0.001 to 5% by weight, but in particular 0.001% by weight to 1% by weight, based on the total weight of the formulation, of the 3,4-dihydroxybenzyl-substituted carbonic acid derivatives according to the invention and may in this connection be in the form of “water-in-oil”, “oil-in-water”, “water-in-oil-in-water” or “oil-in-water-in-oil” emulsions, microemulsions, gels, solutions, e.g. in oils, alcohols or silicone oils, soaps, sticks, aerosols, sprays and also foams. Further customary cosmetic auxiliaries and additives may be present in amounts of 5-99.999% by weight, preferably 10-80% by weight, based on the total weight of the formulation. In addition, the formulations may comprise water in an amount up to 99.999% by weight, preferably 5-80% by weight, based on the total weight of the formulation.

The cosmetic or dermatological preparations according to the invention are prepared using customary processes well known to the person skilled in the art, by incorporating one or more of the 3,4-dihydroxybenzyl-substituted carbonic acid derivatives according to the invention into cosmetic or dermatological formulations which have the customary composition and can be used for the treatment, protection, care and cleansing of the skin, nails or the hair and as make-up products in decorative cosmetics.

To prepare the cosmetic and dermatological preparations according to the invention, in a further embodiment, the 3,4-dihydroxybenzyl-substituted carbonic acid derivatives according to the invention can also be incorporated beforehand into liposomes, e.g. starting from phosphatidylcholine, into microspheres, into nanospheres or else into capsules made of a suitable matrix, e.g. made of natural or synthetic waxes, for example beeswax, carnauba wax, silicone wax or paraffin waxes, and stearyl alcohol, eicosanol, cetyl alcohol, stearin or of gelatin.

For use, the cosmetic and dermatological preparations according to the invention are applied to the skin, the nails and/or the hair in an adequate amount in the manner customary for cosmetics.

The cosmetic and dermatological preparations according to the invention can comprise cosmetic auxiliaries and additives as are customarily used in such preparations, e.g. sunscreens (e.g. organic or inorganic light filter substances, preferably micropigments), preservatives, bactericides, fungicides, virucides, cooling active ingredients, plant extracts, antiinflammatory active ingredients, substances which accelerate wound healing (e.g. chitin or chitosan and derivatives thereof), film-forming substances (e.g. polyvinylpyrrolidones or chitosan or derivatives thereof), customary antioxidants, vitamins (e.g. vitamin C and derivatives, tocopherols and derivatives, vitamin A and derivatives), 2-hydroxycarboxylic acids (e.g. citric acid, malic acid, L-, D-, or dl-lactic acid), skin-lightening agents (e.g. kojic acid, hydroquinone or arbutin), skin-coloring agents (e.g. walnut extracts or dihydroxyacetone), perfumes, antifoams, dyes, pigments which have a coloring action, thickeners, surface-active substances, emulsifiers, emollients, moisturizers and/or humectants (e.g. glycerol or urea), fats, oils, unsaturated fatty acids or derivatives thereof (e.g. linoleic acid, α-linolenic acid, γ-linolenic acid or arachidonic acid and natural or synthetic esters thereof in each case), waxes or other customary constituents of a cosmetic or dermatological formulation, such as alcohols, polyols, polymers, foam stabilizers, electrolytes, organic solvents, silicone derivatives or chelating agents (e.g. ethylenediaminetetraacetic acid and derivatives).

The amounts of amounts of auxiliaries and additives to be used in each case can be readily determined by the person skilled in the art by simple experimentation depending on the nature of the respective product.

In addition to one or more of the 3,4-dihydroxybenzyl-substituted carbonic acid derivatives according to the invention, the preparations according to the invention can additionally also comprise other antioxidants. In particular, other antioxidants which can be used are all antioxidants customary or suitable for cosmetic and/or dermatological applications. The antioxidants are advantageously chosen from the group consisting of amino acids (e.g. glycine, histidine, 3,4-dihydroxyphenylalanine, tyrosine, tryptophan) and derivatives thereof, imidazoles (e.g. urocaninic acid) and derivatives thereof, peptides (D,L-carnosine, D-carnosine, L-carnosine, anserine) and derivatives thereof, carotenoids, carotenes (e.g. α-carotene, β-carotene, lycopene) and derivatives thereof, chlorogenic acid and derivatives thereof, lipoic acid and derivatives thereof, aurothioglucose, propylthiouracil and other thiols (e.g. thioredoxin, glutathione, cysteine, cystine, cystamine and the glycosyl and N-acyl derivatives thereor or alkyl esters thereof), and salts thereof, dilauryl thiodipropionate, distearyl thiodipropionate, thiodipropionic acid and derivatives thereof, and phenolic acid amides of phenolic benzylamines (e.g. homovanillic acid amides, 3,4-dihydroxyphenylacetic acid amides, ferulic acid amides, sinapic acid amides, caffeic acid amides, dihydroferulic acid amides, dihydrocaffeic acid amides, vanillomandelic acid amides or 3,4-dihydroxymandelic acid amides of 3,4-dihydroxybenzylamine, 2,3,4-trihydroxybenzylamine or 3,4,5-trihydroxybenzylamine), catecholoximes or catecholoxime ethers (e.g. 3,4-dihydroxybenzaldoxime or 3,4-dihydroxybenzaldehyde O-ethyloxime), and also (metal) chelating agents (e.g. 2-hydroxy fatty acids, phytic acid, lactoferrin), humic acid, bile acids, bile extracts, bilirubin, biliverdin, folic acid and derivatives thereof, ubiquinone and ubiquinol and derivatives thereof, vitamin C and derivatives thereof (e.g. ascorbyl palmitate, magnesium ascorbyl phosphate, ascorbyl acetate), tocopherols and derivatives (e.g. vitamin E acetate), vitamin A and derivates (e.g. vitamin A palmitat), rutinic acid and derivatives thereof, flavonoids (e.g. quercetin, α-glucosylrutin) and derivatives thereof, phenolic acids (e.g. gallic acid, ferulic acid) and derivatives thereof (e.g. propyl gallate, ethyl gallate, octyl gallate), furfurylideneglucitol, dibutylhydroxytoluene, butylhydroxyanisole, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, resveratrol) and the derivatives of these said active ingredients which are suitable according to the invention.

The amount of further antioxidants in the preparations according to the invention can generally be 0.001 to 30% by weight, preferably 0.001 to 20% by weight, particularly preferably 0.001 to 5% by weight, based on the total weight of the preparation.

Apart from the 3,4-dihydroxybenzyl-substituted carbonic acid derivatives according to the invention, two or more further antioxidants may of course be used.

In the cosmetic or dermatological preparations according to the invention, however, it is also possible to use UV-A and/or UV-B filter substances, where the total amount of filter substances may be 0.1 to 30% by weight, preferably 0.5 to 10% by weight, based on the total weight of the preparations, giving, for example, sunscreens for skin and hair. UV-A and/or UV-B filter substances which may be used are, for example, 3-benzylidenecamphor derivatives (e.g. 3-(4-methyl-benzylidene)-dl-camphor), aminobenzoic acid derivatives (e.g. 2-ethylhexyl 4-(N,N-dimethylamino)benzoate or menthyl anthranilate), 4-methoxycinnamates (e.g. 2-ethylhexyl p-methoxycinnamate or isoamyl p-methoxycinnamate), benzophenones (e.g. 2-hydroxy-4-methoxybenzophenone), mono- or polysulfonated UV filters [e.g. 2-phenylbenzimidazole-5-sulfonic acid, sulisobenzones or 1,4-bis(benzimidazolyl)benzene-4,4′,6,6′-tetrasulfonic acid and 3,3′-(1,4-phenylenedimethylidene)bis(7,7-dimethyl-2-oxobicyclo[2,2,1]heptane-1-methanesulfonic acid) and salts thereof], salicylates (e.g. 2-ethylhexyl salicylate or homomenthyl salicylate), triazines {e.g. 2,4-bis[4-(2-ethylhexyloxy)-2-hydroxyphenyl]-6-(4-methoxyphenyl)-1,3,5-triazine, bis-(2-ethylhexyl) 4,4′-([6-([(1,1-dimethylethyl)aminocarbonyl]-phenylamino)-1,3,5-triazin-2,4-diyl]diimino)bisbenzoate)}, 2-cyanopropenoic acid derivatives (e.g. 2-ethylhexyl 2-cyano-3,3-diphenyl-2-propenoate), dibenzoyl derivatives (e.g. 4-tert-butyl-4′-methoxydibenzoylmethane), polymer-bonded UV filters (e.g. polymers of N-[2-(or 4)-(2-oxo-3-bornylidene)methyl]benzylacrylamide) or pigments (e.g. titanium dioxides, zirconium dioxides, iron oxides, silicon dioxides, manganese oxides, aluminum oxides, cerium oxides or zinc oxides).

The lipid phase in the cosmetic and/or dermatological preparations according to the invention can advantageously be chosen from the following groups of substances: mineral oils (advantageously paraffin oil), mineral waxes, hydrocarbons (advantageously squalane or squalene), synthetic or semisynthetic triglyceride oils (e.g. triglycerides of capric or caprylic acid), natural oils (e.g. castor oil, olive oil, sunflower oil, soybean oil, peanut oil, rapeseed oil, almond oil, palm oil, coconut oil, palm kernel oil, borage seed oil and the like), natural ester oils (e.g. jojoba oil), synthetic ester oils (preferably esters of saturated and/or unsaturated, linear and/or branched alkanecarboxylic acids having 3 to 30 carbon atoms with saturated and/or unsaturated, linear and/or branched alcohols having 3 to 30 carbon atoms and esters of aromatic carboxylic acids with saturated and/or unsaturated, linear and/or branched alcohols having 3 to 30 carbon atoms, in particular chosen from the group consisting of isopropyl myristate, isopropyl stearate, isopropyl palmitate, isopropyl oleate, n-butyl stearate, n-hexyl laurate, n-decyl laurate, isooctyl stearate, isononyl stearate, isononyl isononanoate, 2-ethylhexyl palmitate, 2-ethylhexyl laureate, 2-hexyldecyl stearate, 2-octyldecyl palmitate, oleyl oleate, oleyl erucate, erucyl oleate, erucyl erucate, and synthetic or natural mixtures of such esters), fats, waxes and other natural and synthetic fatty bodies, preferably esters of fatty alcohols with alcohols of low carbon number (e.g. with isopropanol, propylene glycol or glycerol) or esters of fatty alcohols with alkanoic acids of low carbon number or with fatty acids, alkyl benzoates (e.g. mixtures of n-dodecyl, n-tridecyl, n-tetradecyl and n-pentadecyl benzoate), and cyclic or linear silicone oils (such as, for example, dimethylpolysiloxanes, diethylpolysiloxanes, diphenylpolysiloxanes and mixed forms thereof).

The aqueous phase of the cosmetic and/or dermatological preparations according to the invention optionally advantageously comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl ether, monoethyl or monobutyl ether, diethylene glycol monomethyl or monoethyl ether and analogous products, and also alcohols of low carbon number, e.g. ethanol, isopropanol, 1,2-propanediol, glycerol, and also α- or β-hydroxy acids, preferably lactic acid, citric acid or salicylic acid, and also emulsifiers which can advantageously be chosen from the group of ionic, nonionic, polymeric, phosphate-containing and zwitterionic emulsifiers, and in particular one or more thickeners which can advantageously be chosen from the group consisting of silicon dioxide, aluminum silicates, such as, for example, bentonites, polysaccharides and derivatives thereof, e.g. hyaluronic acid, guar kernal flour, xanthan gum, hydroxypropylmethylcellulose or allulose derivatives, particularly advantageously from the group of polyacrylates, preferably a polyacrylate from the group of carbopols, in each case individually or in combination or from the group of polyurethanes.

A particularly advantageous embodiment of the present invention is regarded as being the use of the cosmetic or dermatological preparations according to invention comprising an effective constituent of the 3,4-dihydroxybenzyl-substituted carbonic acid derivatives according to the invention for protecting tissues and cells of mammals, in particular of the skin and/or the hair, against oxidative stress and the harmful effect of free radicals.

The present invention likewise also relates to a process for protecting cosmetic or dermatological preparations, and food and luxury products, against oxidation or photooxidation, the constituents of which are subject to stability problems due to oxidation or photooxidation during storage, characterized in that the preparations have an effective content of 3,4-dihydroxybenzyl-substituted carbonic acid derivatives according to the invention.

### EXAMPLES

Preparation:

### Example 1

### N-(3,4-Dihydroxybenzyl)-O-methylurethane

3,4-Dihydroxybenzylamine hydrochloride (1.5 g, 8.55 mmol) was dissolved in water (15 ml) and 1,4-dioxane (15 ml) and sodium hydrogencarbonate (0.72 g, 8.55 mmol) was added. A solution of methyl chloroformate (0.81 g, 8.55 mmol) in 1,4-dioxane (30 ml) was added dropwise to the mixture. After half of the solution had been added, another portion of sodium hydrogencarbonate (0.81 g, 8.55 mmol) was added. The mixture was then stirred for a further 1.5 h at room temperature, converted to pH 1-2 with 5% strength H₂SO₄, extracted with tert-butyl methyl ether (3×20 ml), the combined organic phase was washed with saturated NaCl solution, dried over Na₂SO₄ and filtered, and the filtrate was concentrated by evaporation at 45° C./20 mbar (2.81 g). The crude product was dissolved in 1,4-dioxane (10 ml) and water (20 ml) saturated NaHCO₃ solution (1 ml) was added and the mixture stirred at 80° C. for 2 h. The reaction mixture was converted to pH 4 with 5% strength H₂SO₄, extracted with tert-butyl methyl ether (3×20 ml), the combined organic phase was washed with saturated NaCl solution, dried over Na₂SO₄ and filtered, and the filtrate was concentrated by evaporation at 50° C./20 mbar (1.89 g). The product was purified over silica gel 60 using the eluent CHCl₃/CH₃OH 7:1 (v/v): 1.34 g of pale brown oil (80% of theory, purity by HPLC>96%); ¹H-NMR (200 MHz, CD₃OD) δ=6.72−6.65 (2H, m), 6.58 (1H, dd, 7 Hz, 2 Hz), 4.11 (2H, s), 3.65 (3H, s) ppm; MS (APCI neg.) m/e=392,66 (100%, [2M-H]⁻), 196.03 (7%, [M-H]⁻).

The compounds below were prepared in accordance with the same procedure:

### Example 2

### N-(3,4-Dihydroxybenzyl)-O-[(1R,3R,4S)menthyl]urethane

MS (APCI pos.) m/e=321.71 (100%, [M+H]⁺), 642.82 (7%, [2M+H]⁺).

### Example 3

### N-(3,4-Dihydroxybenzyl)-O-hexylurethane

¹H-NMR (400 MHz, CD₃OD) δ=6.74−6.68 (2H, m), 6.59 (1H, dd, 7 Hz, 2 Hz), 4.10 (2H, s), 4.03 (2H, t, 6.8 Hz), 1.69−1.56 (2H, m), 1.46−1.25 (6H, m), 0.90 (3H, tm, 6.8 Hz) ppm; MS (APCI neg.) m/e=266.24 (53%, [M-H]⁻), 532.89 (15%, [2M-H]⁻).

### Example 4

### N-(3,4-Dihydroxybenzyl)-O-(2-ethylhexyl)urethane

¹H-NMR (400 MHz, CD₃OD) δ=6.74−6.67 (2H, m), 6.58 (1H, dd, 7 Hz, 2 Hz), 4.12 (2H, s), 3.97 (2H, d, 6.5 Hz), 1.60−1.47 (1H, m), 1.46−1.23 (8H, m), 0.97−0.84 (6H, m) ppm; MS (APCI neg.) m/e=294.77 (42%, [M-H]⁻), 588.92 (32%, [2M-H]⁻).

### Example 5

### N-(3,4-Dihydroxybenzyl)-N′-hexylthiourea

3,4-Dihydroxybenzylamine hydrochloride (400 mg, 2.28 mmol) and triethylamine (460 mg, 4.56 mmol) are suspended in CHCl₃ (15 ml) under nitrogen, and n-hexyl isothiocyanate (343 mg, 2.39 mmol) in CHCl₃ (10 ml) are metered in. The reaction mixture is stirred for a further 18 h at room temperature and then washed with 10% strength hydrochloric acid. The organic phase is washed with water, dried over Na₂SO₄ and filtered, the filtrate is concentrated by evaporation at 40° C./280−20 mbar: 455 mg (71% of theory; purity by HPLC 98%); ¹H-NMR (200 MHz, CD₃OD) δ=6.75 (1H, d, 2 Hz), 6.70 (1H, d, 7.5 Hz), 6.61 (1H, dd, 7.5 Hz, 2 Hz), 4.50 (2H, s, broad), 3.43 (2H, t, broad, ca. 7 Hz), 1.63−1.44 (2H, m), 1.40−1.20 (6H, m), 0.89 (3H, t, ca. 7 Hz) ppm; MS (APCI neg.) m/e=281.70 (100%, [M-H]⁻), 562.90 (29%, [2M-H]⁻).

Preparations:

### Example 6

### “Oil-in-Water” Emulsion

| | | | Content |
|---|---|---|---|
| | Raw material name | | in % by |
| Part | (manufacturer) | Chemical name | wt. |
| A | Arlatone 983 S ® (ICI) | Ether of polyethylene | 1.2 |
| | | glycol with glyceryl | |
| | | monostearate 3, 6, 9, 12, | |
| | | 15, 18, 21, 24, 27, 30, 33, | |
| | Brij 76 ® (ICI) | 36-Decaoxaoctatetracontan- | 1.2 |
| | | 1-ol | |
| | Cutina MD ® (Henkel) | Glyceryl monostearate | 3.5 |
| | Baysilone oil M10 ® | Polydimethylsiloxane | 0.8 |
| | (GE Bayer) | | |
| | Eutanol G ® (Henkel) | Octyldodecanol | 3.0 |
| | Paraffin oil 65 cp (Henry | Mineral oil | 8.0 |
| | Lamotte) | | |
| B | Water, dist. | | 50.35 |
| | | 2-Phenoxyethanol and | |
| | | methyl | |
| | | 4-hydroxybenzoate and | |
| | | ethyl | |
| | Phenonip ® (Nipa | 4-hydroxybenzoate and | 0.5 |
| | Laboratories) | propyl | |
| | | 4-hydroxybenzoate and | |
| | | butyl | |
| | | 4-hydroxybenzoate | |
| | 1,2-Propylene glycol | | 2.0 |
| | Glycerol 99% | | 3.0 |
| | Trilon BD | Disodium EDTA | 0.1 |
| | N-(3,4-Dihydroxybenzyl)- | | 0.1 |
| | O-methylurethane | | |
| | (Example 1) | | |
| C | Water, dist. | | 25.0 |
| | Carbopol 2050 ® (B.F. | Crosslinked acrylic acid/ | 0.4 |
| | Goodrich) | C₁₀-C₃₀-alkyl acrylate | |
| | | polymer | |
| | Aqueous sodium hydroxide | | 0.85 |
| | solution, 10% | | |

Part A was mixed and heated to 80° C. Part B was mixed and heated to 90° C. and added to part A with stirring. For part C, Carbopol was carefully dispersed in water and neutralized with sodium hydroxide solution (pH 5.4). Part C was then added to the mixture of parts A and B at 60° C.

### Example 7

### Butylene glycol Solution

| | Content |
|---|---|
| | in % by |
| Raw material name | wt. |
| 1,3-Butylene glycol | 99.9 |
| N-(3,4-Dihydroxybenzyl)-O-methylurethane | 0.1 |
| (Example 1) | |

### Example 8

### “Water-in-Oil” Sunscreen Emulsion with UVA/B Broadband Protection

| | | | Content |
|---|---|---|---|
| | Raw material name | | in % by |
| Part | (manufacturer) | Chemical name | wt. |
| A | Dehymuls PGPH ® | Polyglycerol-2 | 3.0 |
| | (Henkel) | dipolyhydroxystearate | |
| | Monomuls 90-O 18 ® | Glyceryl oleate | 1.0 |
| | (Henkel) | | |
| | Permulgin 2550 ® | Beeswax | 1.0 |
| | (Koster Keunen Holland) | | |
| | Myritol 318 ® (Henkel) | Caprylic/caproic | 6.0 |
| | | triglycerides | |
| | Witconol TN ® (Witco) | C₁₂-C₁₅-Alkylbenzoate | 6.0 |
| | Cetiol SN ® (Henkel) | Cetyl and stearyl | 5.0 |
| | | isononanoate | |
| | Copherol 1250 ® (Henkel) | Tocopherol acetate | 1.0 |
| | Solbrol P ® (Bayer) | Propyl 4-hydroxybenzoate | 0.1 |
| | Neo Heliopan ® AV | 2-Ethylhexyl p-methoxy- | 4.0 |
| | (Haarmann & Reimer) | cinnamate | |
| | Neo Heliopan ® E 1000 | Isoamyl | 4.0 |
| | (Haarmann & Reimer) | p-methoxycinnamate | |
| | Neo Heliopan ® MBC | 3-(4-Methylbenzylidene)- | 2.0 |
| | (Haarmann & Reimer) | dl-camphor | |
| | Neo Heliopan ® OS | 2-Ethylhexyl salicylate | 3.0 |
| | (Haarmann & Reimer) | | |
| | Octyltriazone | | 1.0 |
| | Zinc oxide neutral | | 7.0 |
| | (Haarmann & Reimer) | | |
| B | Water, dist. | | 40 |
| | Phenoxyethanol | | 0.7 |
| | Solbrol M (Bayer) | Methyl 4-hydroxybenzoate | 0.2 |
| | Glycerol 99% | | 4.0 |
| | Neo Heliopan ® Hydro | 2-Phenylbenzimidazole-5- | 10.0 |
| | (Haarmann & Reimer), | sulfonic acid | |
| | 15% as sodium salt | | |
| | Benzophenone-4 | | 0.5 |
| | N-(3,4-Dihydroxybenzyl)- | | 0.1 |
| | O-methylurethane | | |
| | (Example 1) | | |
| C | Perfume oil | | 0.3 |
| | Bisabolol | | 0.1 |

For part A, all the substances apart from the zinc oxide were heated to 85° C. and the zinc oxide was carefully dispersed in the mixture. The components of part B were mixed, heated to 85° C. and added to part A with stirring. Part C was added to the mixture of parts A and B and then the mixture was homogenized with a dispersion tool.

### Example 9

### “Oil-in-Water” Sunscreen Emulsion with UVA/B Broadband Protection

| | | | Content |
|---|---|---|---|
| | Raw material name | | in % by |
| Part | (manufacturer) | Chemical name | wt. |
| A | Arlacel 165 ® (ICI) | Glyceryl stearate and | 3.0 |
| | | polyethylene glycol 100 | |
| | | stearate | |
| | Emulgin B2 ® (Henkel) | Ceteareth-20 | 1.0 |
| | Lanette O ® (Henkel) | Cetyl and stearyl alcohol | 1.15 |
| | Myritol 318 ® (Henkel) | Caprylic/caproic | 5.0 |
| | | triglycerides | |
| | Cetiol SN ® (Henkel) | Cetyl and stearyl | 4.0 |
| | | isononanoate | |
| | Abil 100 ® (Goldschmidt) | Polydimethylsiloxane | 1.0 |
| | Bentone Gel MIO ® | Mineral oil and quaternium- | 3.0 |
| | (Rheox) | 18 hectorite and propylene | |
| | | carbonate | |
| | Cutina CBS ® (Henkel) | Glyceryl stearate and cetyl | 2.0 |
| | | alcohol and stearyl alcohol | |
| | | and cetyl palmitate and | |
| | | cocoglycerides | |
| | Neo Heliopan ® 303 | 2-Ethylhexyl 2-cyano-3,3- | 7.0 |
| | (Haarmann & Reimer) | diphenyl-2-propenoate | |
| | Neo Heliopan ® BB | 2-Hydroxy-4- | 1.0 |
| | (Haarmann & Reimer) | methoxybenzo-phenone | |
| | Neo Heliopan ® MA | Menthyl anthranilate | 3.0 |
| | (Haarmann & Reimer) | | |
| | 2-Ethylhexyl N,N- | | 3.0 |
| | dimethyl-4-aminobenzoate | | |
| | Titanium dioxide microfine | | 5.0 |
| B | Water, dist. | | 55.85 |
| | Veegum ultra ® | Magnesium aluminum | 1.0 |
| | (Vanderbilt) | sulfate | |
| | Natrosol 250 HHR | Hydroxymethylcellulose | 0.3 |
| | (Hercules) | | |
| | Glycerol | | 3.0 |
| | | 2-Phenoxyethanol and | |
| | | methyl | |
| | | 4-hydroxybenzoate and | |
| | | ethyl | |
| | Phenonip ® (Nipa | 4-hydroxybenzoate and | 0.3 |
| | Laboratories) | propyl | |
| | | 4-hydroxybenzoate and | |
| | | butyl | |
| | | 4-hydroxybenzoate | |
| | N-(3,4-Dihydroxybenzyl)- | | 0.1 |
| | O-methylurethane | | |
| | (Example 1) | | |
| C | Perfume oil | | 0.3 |

For part A, all of the substances apart from the titanium dioxide were mixed and heated to 85° C.; the titanium dioxide was carefully dispersed into the mixture. For part B, all of the substances apart from Veegum and Natrosol were mixed, heated to 90° C., Natrosol and Veegum were dispersed therein and the mixture was added to part A with stirring. Part C was added to the mixture of parts A and B and then the mixture was homogenized using a dispersion tool (pH 5.6).

### Example 10

### “Oil-in-Water” Sunscreen Emulsion with UVA/B Broadband Protection

| | | | Content |
|---|---|---|---|
| | Raw material name | | in % by |
| Part | (manufacturer) | Chemical name | wt. |
| A | Crodaphos MCA ® | Cetyl phosphate | 1.50 |
| | (Croda) | | |
| | Cutina MD ® (Henkel) | Glyceryl stearate | 2.0 |
| | Lanette 16 ® (Henkel) | Cetyl alcohol | 1.2 |
| | Myritol 318 ® (Henkel) | Caprylic/caproic | 5.0 |
| | | triglycerides | |
| | Cetiol SN ® (Henkel) | Cetyl and stearyl | 5.0 |
| | | isononanoate | |
| | Copherol 1250 ® (Henkel) | Tocopherol acetate | 0.5 |
| | Solbrol P ® (Bayer) | Propyl 4-hydroxybenzoate | 0.1 |
| | N-(3,4-Dihydroxybenzyl)- | | 0.1 |
| | O-n-hexylurethane | | |
| | (Example 3) | | |
| | Abil 100 ® (Goldschmidt) | Polydimethylsiloxane | 0.3 |
| | Neo Heliopan ® HMS | 3,3,5-Trimethylcyclohexyl | 5.0 |
| | (Haarmann & Reimer) | salicylate | |
| | Neo Heliopan ® 357 | Butylmethoxydibenzoyl- | 2.0 |
| | (Haarmann & Reimer) | methane | |
| B | Water, dist. | | 47.8 |
| | 1,3-Butylene glycol | | 3.0 |
| | Sobrol M ® (Bayer) | Methyl 4-hydroxybenzoate | 0.2 |
| | Phenoxyethanol | | 0.7 |
| | Carbopol ETD 2050 ® | Copolymer acrylic acid/ | 0.2 |
| | (B. F. Goodrich) | C₁₀-C₃₀-alkyl acrylate | |
| | Keltrol T ® (Calgon) | Xanthan gum | 0.2 |
| | Neo Heliopan ® AP | 2,2-(1,4-Phenylene)-bis- | 22 |
| | (Haarmann & Reimer) | (1H-benzimidazole-4,6- | |
| | | disulfonic acid) and | |
| | | disodium salt | |
| C | Aqueous sodium hydroxide | | 2.8 |
| | solution, 10% | | |
| D | Perfume oil | | 0.3 |
| | Bisabolol | | 0.1 |

Part A was heated to 85° C. Part B: Carbopol and Keltrol were dispersed into the remaining constituents in the cold, the mixture was heated to 85° C. and added to part A. Part C was immediately added to the mixture of parts A and B at 80° C. and homogenized for 5 min with a dispersion tool. Finally, part D was added at room temperature and the mixture was homogenized using a dispersion tool (pH 6.6).

Use:

### Example 11

### Activity as Free-Radical Scavengers

The activity of the exemplary compounds as free-radical scavengers was compared with that of conventional free-radical scavengers. For this purpose, the DPPH-(1,1-diphenyl-2-picrylhydrazyl) test for the removal of free radicals was used.

DPPH was dissolved in methanol to a concentration of 100 μmol/l. A series of dilutions of the exemplary compounds, vitamin C, α-tocopherol and 3,5-di-tert-butyl-4-hydroxytoluene were prepared in methanol. Methanol served as the control. 2500 μl of the DPPH solution were mixed with 500 μl of each test solution and the decrease in absorption at 515 nm was read until the decrease was less than 2% per hour. The activity of the test substances as free-radical scavengers was calculated according to the following equation:

Activity as free-radical scavenger (%)=100−(absorption of the test compounds)/(absorption of the control)×100.

The activity as free-radical scavenger (%) in a series of dilutions of test compounds was used to calculate, for each test compound, the effective relative concentration EC₅₀ (based on the starting concentration of DPPH, EC=c(test compound)/c(DPPH)) of a test compound at which 50% of the free radical DPPH had been removed. The results are given in Table 2:

TABLE 2

| Test compound (Example number) | EC₅₀/(mol/mol) |
|---|---|
| N-(3,4-Dihydroxybenzyl)-O-methylurethane(1) | 0.29 |
| N-(3,4-Dihydroxybenzyl)-O-[(1R,3R,4S)menthyl] | 0.36 |
| urethane (2) | |
| N-(3,4-Dihydroxybenzyl)-O-hexylurethane (3) | 0.19 |
| N-(3,4-Dihydroxybenzyl)-O-(2-ethylhexyl)urethane (4) | 0.13 |
| N-(3,4-Dihydroxybenzyl)-N′-(n-hexyl)thiourea (5) | 0.06 |
| Vitamin C | 0.27 |
| α-Tocopherol | 0.25 |
| 3,5-Di-tert-butyl-4-hydroxytoluene | 0.24 |

### Example 12

### Activity as Antioxidants

The activity of the exemplary compounds as antioxidants was compared with that of conventional antioxidants. The test system used was the accelerated autoxidation of lipids by air with or without antioxidant using the Rancimat apparatus (Rancimat is a registered trademark of Metrohm AG, Herisau, Switzerland).

The exemplary compounds, vitamin C, α-tocopherol and 3,5-di-tert-butyl-4-hydroxytoluene were dissolved in methanol or acetone, and 100 μl of each test solution was added to a 3 g prepared oil sample. In a control sample, only solvent was added. A constant stream of dry air (20 l/h) was bubbled through the heated oil sample which contained the test solution, and the volatile oxidation products (predominantly short-chain fatty acids such as formic acid or acetic acid) were collected in a receiver containing water. The conductivity of this aqueous solution was continuously measured and documented. The oxidation of (unsaturated) fats proceeds only very slowly for some time and then suddenly increases. The time to the increase is referred to as the induction period (IP).

The following equation was used to calculate the antioxidative index (AOI):

AOI=IP_{(with test solution)}/IP_{(control sample)}.

The results for the experiment at 100° C. in soybean oil that has been purified over alumina grade N are given in Table 3:

TABLE 3

| | AOI in soybean |
|---|---|
| | oil at 100° C. |
| | with 0.05% test |
| Test compound (Example number) | substance |
| N-(3,4-Dihydroxybenzyl)-O-methylurethane (1) | 7.2 |
| N-(3,4-Dihydroxybenzyl)-O-[(1R,3R,4S)menthyl] | 6.5 |
| urethane (2) | |
| N-(3,4-Dihydroxybenzyl)-O-hexylurethane (3) | 14.5 |
| N-(3,4-Dihydroxybenzyl)-O-(2-ethylhexyl)urethane (4) | 10.7 |
| Vitamin C | 1.2 |
| α-Tocopherol | 5.1 |
| 3,5-Di-tert-butyl-4-hydroxytoluene | 4.8 |

The results for the experiment at 80° C. in squalene that has been purified over alumina grade N and stabilized with 1 ppm of α-tocopherol are given in Table 4:

TABLE 4

| | AOI in squalene |
|---|---|
| | at 80° C. with |
| | 0.005% test |
| Test compound (example number) | substance |
| N-(3,4-Dihydroxybenzyl)-O-methylurethane (1) | 34 |
| N-(3,4-Dihydroxybenzyl)-O-[(1R,3R,4S)menthyl] | 48 |
| urethane (2) | |
| N-(3,4-Dihydroxybenzyl)-O-hexylurethane (3) | 49 |
| N-(3,4-Dihydroxybenzyl)-O-(2-ethylhexyl)urethane (4) | 45 |
| N-(3,4-Dihydroxybenzyl)-N′-(n-hexyl)thiourea (5) | 62 |
| Vitamin C | 0.7 |
| α-Tocopherol | 39 |
| 3,5-Di-tert-butyl-4-hydroxytoluene | 38 |

### Example 13

### Determination of the Protective Action Against Ultraviolet Light-Induced Sebum Oxidation

A 0.2% strength ethanolic solution of tocopherol was prepared as internal standard (solution A). A dose of 2 mg/cm² of the formulation from Example 7 was applied twice daily to the skin on the backs of 12 subjects for 2 days in each case. Prior to the subsequent irradiation, solution A was applied to a control area (2 mg/cm²). The 2 treated and one untreated sites were irradiated with ultraviolet light (320 to 400 nm, 10 joules/cm²). The test areas in each case were treated with 4 ml of ethanol for 2 min, the solutions were dried under nitrogen at room temperature and the residue was taken up in 1 ml of ethanol. The latter solutions were investigated by HPLC for their content of squalene (detection at 210 nm against standard) or squalene hydroperoxide (SQOOH, determination of the peroxide content by means of cytochrome C/luminol-enhanced chemiluminescence). The content of squalene peroxide was given relative to squalene in the form of picomoles of peroxide per μg of squalene.

The inhibition based on the untreated areas was calculated using the following equation:

% inhibition=100·(c_{SQOOH, untreated}−c_{SQOOH, treated})/C_{SQOOH, untreated}

| | c(H₂O₂)/c(SQ) | % inhibition compared |
|---|---|---|
| Test compound | [pmol/μg] | with the untreated area |
| Untreated area | 930 ± 65 | — |
| Formulation from Example 7 | 565 ± 35 | 40 ± 6 |
| Solution A | 664 ± 19 | 28 ± 6 |

1. A 3,4-dihydroxybenzyl-substituted carbonic acid derivative of the general formula I, 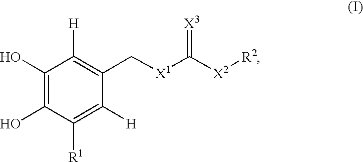
where
X¹, X² and X³, independently of one another, are oxygen atoms, sulfur atoms or groups —N—R³,
and
R¹ is a hydrogen atom, a lower alkyl, lower alkenyl, 1-oxo-lower alkyl or 1-oxo-lower alkenyl group having 1 to 5 carbon atoms or a group —O—R⁴, in which R⁴ is a hydrogen atom, a lower alkyl, lower alkenyl, 1-oxo-lower alkyl or 1-oxo-lower alkenyl group having 1 to 5 carbon atoms,
and
R² is a hydrogen atom, a branched or unbranched, cyclic or stretched alkyl or alkenyl group having 1 to 22 carbon atoms, where one or more of the carbon atoms may be replaced by oxygen atoms, or a ring-substituted arylalkyl group having 7 to 15 carbon atoms, with the proviso that the ring-substituted arylalkyl group does not contain halogen atoms,
and
R³ is a hydrogen atom, a branched or unbranched, cyclic or stretched alkyl or alkenyl group having 1 to 22 carbon atoms, where one or more of the carbon atoms may be replaced by oxygen atoms.

2. The 3,4-dihydroxybenzyl-substituted carbonic acid derivative as claimed in claim 1 of the general formula (Ia), 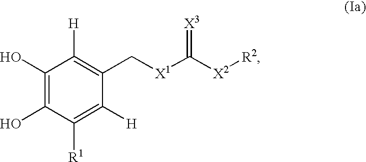
where
R¹ is a hydrogen atom, a lower alkyl, lower alkenyl, 1-oxo-lower alkyl or 1-oxo-lower alkenyl group having 1 to 5 carbon atoms or a group —O—R⁴, in which R⁴ is a hydrogen atom, a lower alkyl, lower alkenyl, 1-oxo-lower-alkyl or 1-oxo-lower-alkenyl group having 1 to 5 carbon atoms,
and
X¹ is —NH, and
X² and X³ are oxygen, and
R² is a hydrogen atom, a methyl, an ethyl or an ethenyl group.

3. The 3,4-dihydroxybenzyl-substituted carbonic acid derivative as claimed in claim 1 or 2 of the general formula 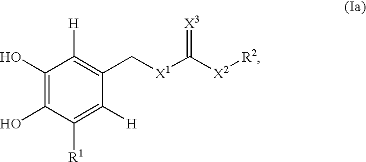
where
R¹ is a hydrogen atom, a lower alkyl group having 1 to 5 carbon atoms or a group —O—R⁴, in which R⁴ is a hydrogen atom or a lower alkyl group having 1 to 5 carbon atoms,
and
X¹ is —NH, and
X² and X³ are oxygen, and
R² is a hydrogen atom, a methyl, an ethyl or an ethenyl group.

4. The 3,4-dihydroxybenzyl-substituted carbonic acid derivative as claimed in claims 1 to 3 of the general formula (Ia),
where
R¹ is a hydrogen atom, a methoxy or a hydroxyl group,
and
X¹ is —NH, and
X² and X³ are oxygen, and
R² is a methyl, an ethyl or an ethenyl group.

5. The 3,4-dihydroxybenzyl-substituted carbonic acid derivative as claimed in claim 1 of the general formula (Ib), 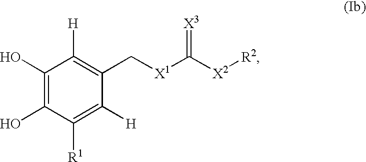
where
R¹ is a hydrogen atom, a lower alkyl, lower alkenyl, 1-oxo-lower alkyl or 1-oxo-lower alkenyl group having 1 to 5 carbon atoms or a group —O—R⁴, in which R⁴ is a hydrogen atom, a lower alkyl, lower alkenyl, 1-oxo-lower alkyl or 1-oxo-lower alkenyl group having 1 to 5 carbon atoms,
and
X¹ is —NH, and
X² and X³ are oxygen, and
R² is a branched or unbranched, cyclic or stretched alkyl or alkenyl group having 3 to 22 carbon atoms, where one or more of the carbon atoms may be replaced by oxygen atoms, or a ring-substituted arylalkyl group having 7 to 15 carbon atoms, with the proviso that the ring-substituted arylalkyl group does not contain halogen atoms.

6. The 3,4-dihydroxybenzyl-substituted carbonic acid derivative as claimed in claim 1 or 5 of the general formula 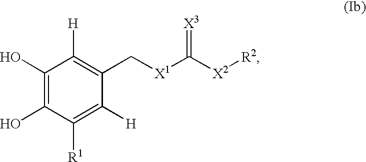
where
R¹ is a hydrogen atom, a lower alkyl group having 1 to 5 carbon atoms or a group —O—R⁴, in which R⁴ is a hydrogen atom or a lower alkyl group having 1 to 5 carbon atoms,
and
X¹ is —NH, and
X² and X³ are oxygen, and
R² is a branched or unbranched, cyclic or stretched alkyl or alkenyl group having 3 to 20 carbon atoms, where one or more of the carbon atoms may be replaced by oxygen atoms, or a ring-substituted arylalkyl group having 7 to 8 carbon atoms, with the proviso that the ring-substituted arylalkyl group does not contain halogen atoms.

7. The 3,4-dihydroxybenzyl-substituted carbonic acid derivative as claimed in claim 1, 6 or 7 of the general formula 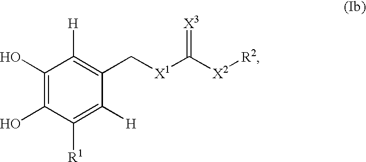
where
R¹ is a hydrogen atom, a methoxy or a hydroxyl group,
and
X¹ is —NH, and
X² and X³ are oxygen, and
R² is a branched or unbranched, cyclic or stretched alkyl group having 3 to 18 carbon atoms, a branched or unbranched, cyclic or stretched alkenyl group having 3 to 18 carbon atoms or a ring-substituted benzyl, ring-substituted 2-phenylethyl or ring-substituted 1-phenylethyl radical, with the proviso that the ring-substituted arylalkyl group does not contain halogen atoms.

8. The 3,4-dihydroxybenzyl-substituted carbonic acid derivative as claimed in claim 1 of the general formula (Ic), 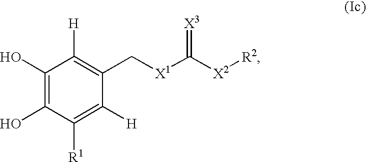
where
R¹ is a hydrogen atom, a lower alkyl, lower alkenyl, 1-oxo-lower alkyl or 1-oxo-lower alkenyl group having 1 to 5 carbon atoms or a group —O—R⁴, in which R⁴ is a hydrogen atom, a lower alkyl, lower alkenyl, 1-oxo-lower alkyl or 1-oxo-lower alkenyl group having 1 to 5 carbon atoms,
X¹, X² and X³, independently of one another, are oxygen atoms, sulfur atoms or groups —NH, with the proviso that X¹ is not —NH at the same time as X² and X³ are oxygen,
and
R² is a branched or unbranched, cyclic or stretched alkyl or alkenyl group having 1 to 22 carbon atoms, where one or more of the carbon atoms may be replaced by oxygen atoms, or a ring-substituted arylalkyl group having 7 to 15 carbon atoms, with the proviso that the ring-substituted arylalkyl group does not contain halogen atoms.

9. The 3,4-dihydroxybenzyl-substituted carbonic acid derivative as claimed in claim 1 or 8 of the general formula (Ic), 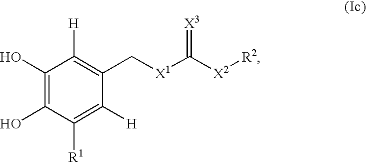
where
X¹ and X², independently of one another, are oxygen atoms, sulfur atoms or —N—H,
and
X³ is an oxygen atom or a sulfur atom, with the proviso that X¹ is not —NH at the same time as X² and X³ are oxygen
and
R is a hydrogen atom, a lower alkyl group having 1 to 5 carbon atoms or a group —O—R⁴, in which R⁴ is a hydrogen atom or a lower alkyl group having 1 to 5 carbon atoms,
and
R² is a branched or unbranched, cyclic or stretched alkyl or alkenyl group having 1 to 20 carbon atoms, where one or more of the carbon atoms may be replaced by oxygen atoms, or a ring-substituted arylalkyl group having 7 to 8 carbon atoms, with the proviso that the ring-substituted arylalkyl group does not contain halogen atoms.

10. The 3,4-dihydroxybenzyl-substituted carbonic acid derivative as claimed in claim 1, 8 or 9 of the general formula (Ic), 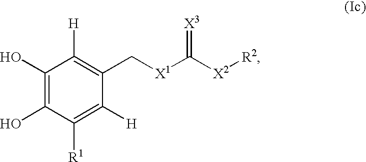
where
R¹ is a hydrogen atom, a methoxy or a hydroxyl group,
and
X¹ is a group —N—H, and
X² is an oxygen atom, sulfur atom or —N—H, and
X³ is an oxygen atom or a sulfur atom, with the proviso that X² and X³ are not oxygen at the same time, and
R² is a branched or unbranched, cyclic or stretched alkyl group having 1 to 18 carbon atoms, a branched or unbranched, cyclic or stretched alkenyl group having 2 to 18 carbon atoms or a ring-substituted benzyl, ring-substituted 2-phenylethyl or ring-substituted 1-phenylethyl radical, with the proviso that the ring-substituted arylalkyl group does not contain halogen atoms.

11. The 3,4-dihydroxybenzyl-substituted carbonic acid derivative as claimed in claims 1 to 10, characterized in that they are chosen from the group consisting of N-(3,4-dihydroxybenzyl)-O-methylurethane, N-(3,4-dihydroxybenzyl)-O-[(1R,3R,4S)-menthyl]urethane, O-methyl-N-(3,4,5-trihydroxybenzyl)-urethane, N-(3,4-dihydroxybenzyl)-O-hexylurethane, N-(3,4-dihydroxy-benzyl)-O-(2-ethylhexyl)urethane, N-(3,4-dihydroxybenzyl)-N′-hexylthiourea and N,N′-bis(3,4-dihydroxybenzyl)urea.

12. The 3,4-dihydroxybenzyl-substituted carbonic acid derivative of the general formula (I) as claimed in claims 1 to 11 in the form of its tautomers.

13. A process for the preparation of 3,4-dihydroxybenzyl-substituted carbonic acid derivatives of the general formula (I), characterized in that a 3,4-dihydroxybenzyl derivative of the general formula (II) 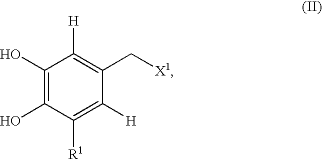
where
X¹ is a group —O—H, —S—H, —NH₂ 2 or —(NH₃)⁺ and
R¹ has the meaning given above
either
with an activated carbonic acid derivative of the general fomula (III) 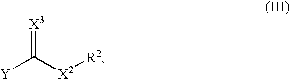
where
X², X³ and R² have the meaning given above and
Y is a halogen atom, a group —N₃, —O—N═C(C₆H₅)CN, —O—R⁵ or —S—R⁵, and
R⁵ may be an alkyl, alkyl-1-oxo, alkenyl, alkenyl-1-oxo, aryl, arylalkyl, arylalkyl-1-oxo or alkyloxycarbonyl group,
or
with a heterocumulene of the general formula (IV)
X⁴═C═N—R² (IV),
where
X⁴ is an oxygen atom or a sulfur atom and
R² has the meaning given above,
or
with phosgene or triphosgene with or without solvent and optionally with the admixing of an auxiliary base and either directly after purification with a compound of the general formula (V)
Z-R² (V),
where Z is a group —O—H, —S—H, —NH₂ or —(NH₃)⁺ and
R² has the meaning given above
with or without solvent and optionally with the admixing of an auxiliary base.

14. The process as claimed in claim 13, characterized in that the 3,4-dihydroxybenzyl derivatives of the general formula (II) used are 3,4-dihydroxybenzylamine or ammonium salts thereof, 3,4-dihydroxy-5-methylbenzylamine or ammonium salts thereof, 3,4-dihydroxy-5-methoxy-benzylamine or ammonium salts thereof, 3,4,5-trihydroxybenzylamine or ammonium salts thereof, 3,4-dihydroxybenzyl alcohol or 3,4,5-trihydroxybenzyl alcohol.

15. The process as claimed in claim 13 or 14, characterized in that the activated carbonic acid derivatives of the general formula (III) used are methyl chloroformate, ethyl chloroformate, propyl chloroformate, isopropyl chloroformate, n-butyl chloroformate, chloroformic acid, 2-(tert-butoxycarbonyloxyimino)-2-phenylacetonitrile, di-tert-butyl pyrocarbonate, (−)-menthyl chloroformate, n-hexyl chloroformate, 2-ethylhexyl chloroformate or other chloroformic esters of branched or unbranched, stretched or cyclic alkanols or alkenols.

16. The process as claimed in claims 13 to 15, characterized in that the heterocumulenes of the general formula (IV) used are methyl, ethyl, propyl, isopropyl, n-butyl, isobutyl, sec-butyl, tert-butyl, the various stretched or cyclic isomers of pentyl, hexyl, heptyl and octyl isothiocyanates or the corresponding isocyanates.

17. The process as claimed in claims 13 to 16, characterized in that the compounds of the general formula (V) used are methyl-, ethyl-, propyl-, isopropyl-, n-butyl-, isobutyl-, sec-butyl-, tert-butyl-, the various stretched or cyclic isomers of pentyl-, hexyl-, heptyl-, ring-substituted benzyl-, 1-phenylethyl- and 2-phenylethylamines, alcohols or thiols.

18. The use of the 3,4-dihydroxybenzyl-substituted carbonic acid derivatives as claimed in claims 1 to 12 as antioxidants and/or free-radical scavengers.

19. The use of the 3,4-dihydroxybenzyl-substituted carbonic acid derivatives as claimed in claims 1 to 12 for protecting cells and tissues of humans against the harmful effects of free radicals and reactive oxygen compounds which accelerate aging.

20. The use of the 3,4-dihydroxybenzyl-substituted carbonic acid derivatives as claimed in claims 1 to 12 for protecting cosmetic or dermatological preparations against oxidation or photooxidation.

21. The use of the 3,4-dihydroxybenzyl-substituted carbonic acid derivatives as claimed in claims 1 to 12 and 18 to 20 in combination with other antioxidants or free-radical scavengers.

22. The use of the 3,4-dihydroxybenzyl-substituted carbonic acid derivatives as claimed in claims 1 to 12 and 18 to 21, for protecting foods and luxury products.

23. A cosmetic preparation comprising 0.001% by weight to 10% by weight of the 3,4-dihydroxybenzyl-substituted carbonic acid derivatives as claimed in claims 1 to 12, based on the total weight of the preparation.

24. A dermatological preparation comprising 0.001% by weight to 10% by weight of the 3,4-dihydroxybenzyl-substituted carbonic acid derivatives as claimed in claims 1 to 12, based on the total weight of the preparation.

25. A cosmetic preparation comprising 0.001% by weight to 10% by weight of the 3,4-dihydroxybenzyl-substituted carbonic acid derivatives as claimed in claims 1 to 12, based on the total weight of the preparation, and at least one further UVA and/or UVB filter substance.

26. A nail care preparation comprising 0.001% by weight to 10% by weight of the 3,4-dihydroxybenzyl-substituted carbonic acid derivatives as claimed in claims 1 to 12, based on the total weight of the preparation.

27. A hair care preparation comprising 0.001% by weight to 10% by weight of the 3,4-dihydroxybenzyl-substituted carbonic acid derivatives as claimed in claims 1 to 12, based on the total weight of the preparation.

28. The cosmetic or dermatological preparation as claimed in claims 22 to 27 which, in addition to the 3,4-dihydroxybenzyl-substituted carbonic acid derivatives as claimed in claims 1 to 12, comprises at least one further antioxidant.

29. A food and/or luxury product comprising 0.001% by weight to 10% by weight of the 3,4-dihydroxybenzyl-substituted carbonic acid derivatives as claimed in claims 1 to 12 based on the total weight of the preparation.

30. The food and/or luxury product as claimed in claim 29, which, in addition to the 3,4-dihydoxybenzyl-substituted carbonic acid derivatives as claimed in claims 1 to 12, comprises at least one further antioxidant.

31. The use of the preparations as claimed in claims 22 to 30 for protecting cells and tissue of humans against the harmful effects of free radicals and reactive oxygen compounds which accelerate aging.
